(19) United States
(12) Patent Application Publication
(10) Pub. No.: US 2003/0060380 A1
(43) Pub. Date: Mar. 27, 2003

(54) Bio-compatible remover composition for removing medical grade and other adhesives, and kit including the same

(75) Inventors: Jaime Ayarza, Wake Forest, NC (US); Ibraheem T. Badejo, Morrisville, NC (US); Joe B. Barefoot, Raleigh, NC (US); Daniel L. Hedgpeth, Raleigh, NC (US); Michelle M. Knotts, Raleigh, NC (US); Bernard Malofsky, Bloomfield, CT (US); Upvan Narang, Raleigh, NC (US); Gina L. Spath, Wake Forest, NC (US); Wendy Y. Su, Cary, NC (US)

Correspondence Address: OLIFF & BERRIDGE, PLC, P.O. BOX 19928, ALEXANDRIA, VA, 22320, US

(73) Assignee: Closure Medical Corporation, Raleigh, NC (US)

(21) Appl. No.: 09/962,268

(22) Filed: Sep. 26, 2001

(51) Int. Cl. A61K 7/48; A61K 7/50

(52) U.S. Cl. 510/134; 510/159; 510/319; 510/466; 510/488

(57) ABSTRACT

An adhesive remover composition and methods for removing medical grade and other adhesive compositions including, but not limited to, cyanoacrylate adhesive compositions from surfaces including, but not limited to, areas of tissue, including areas of compromised skin. The composition, which is preferably bio-compatible, includes a suitable remover compound such as an alkylester plasticizer combined with an antimicrobial preservative to protect the composition from microorganisms. A method of removing adhesive compositions includes applying the remover composition to the adhesive composition using an applicator, such as a spray applicator or a towelette, allowing the remover to interact with the adhesive composition and then removing the remover and the adhesive composition.

### BACKGROUND OF THE INVENTION

1. Field of Invention

The invention relates to a bio-compatible remover composition for removing medical grade and other monomer and polymer adhesive and sealant compositions, and to the production and use of such a remover for industrial and medical applications. The present invention also relates to a kit including such a remover composition.

2. Description of Related Art

Monomer and polymer adhesives are used in both industrial (including household) and medical applications. Included among these adhesives are the 1,1-disubstituted ethylene monomers and polymers, such as the α-cyanoacrylates. Since the discovery of the adhesive properties of such monomers and polymers, they have found wide use due to the speed with which they cure, the strength of the resulting bond formed, and their relative ease of use. These characteristics have made the α-cyanoacrylate adhesives the primary choice for numerous applications such as bonding plastics, rubbers, glass, metals, wood, and, more recently, biological tissues.

It is known that monomeric forms of α-cyanoacrylates are extremely reactive, polymerizing rapidly in the presence of even minute amounts of an initiator, including moisture present in the air or on moist surfaces such as animal (including human) tissue. Monomers of α-cyanoacrylates are anionically polymerizable or free radical polymerizable, or polymerizable by zwitterions or ion pairs to form polymers. Once polymerization has been initiated, the cure rate can be very rapid.

Medical applications of 1,1-disubstituted ethylene adhesive compositions include use as an alternate or an adjunct to surgical sutures and/or staples in wound closure, as well as for covering and protecting surface wounds such as lacerations, abrasions, burns, stomatitis, sores, minor cuts and scrapes, and other wounds. When an adhesive is applied to surfaces to be joined, it is usually applied in its monomeric form, and the resultant polymerization gives rise to the desired adhesive bond.

U.S. Pat. Nos. 5,514,371, 5,514,372, 5,575,997, 5,624,669, and 5,582,834 to Leung et al. disclose the addition of a therapeutic agent in a cyanoacrylate composition. The cyanoacrylate adhesive forms a matrix for the therapeutic agent, with the therapeutic agent being released in vivo over time from the matrix during biodegradation of the polymer.

U.S. Pat. No. 5,762,955 to Smith discloses a treatment for healthy, damaged, diseased, or infected biological tissue by applying a bioadhesive coating in conjunction with a medication. The treatment is directed, in part, to treating external biological tissue that may be affected by harmful afflictions such as bruises, bums, dermatological afflictions, infections, gashes, wounds, herpes sores, canker sores, or intra-oral lesions, and skin cancers. Smith further discloses several medications that may be used including corticosteroids, fluoroouracil, obtundants, anesthetics, antibiotics, fungicides, anti-inflammatory agents, antibacterial agents, antiseptic agents, and other medications or combinations of medications used in processes for healing tissue, promoting or preventing blood clotting, destroying cancer cells, palliative treatments and killing of bacteria or viruses.

U.S. Pat. No. 4,880,416 to Horiuchi et al., discloses a dermal bandage of a pre-formed film-like adhesive material for preventing dermally applied ointments, creams, solutions, powders, etc. from falling off, and for delivering drugs, such as anti-fungal agents, to affected parts of the skin. U.S. Pat. Nos. 5,716,607 and 5,716,608, both to Byram et al., disclose the use of cyanoacrylate adhesives to prevent ionization radiation damage to skin. Such damage is prevented by applying the cyanoacrylate polymer to the skin to be protected. U.S. Pat. No. 5,653,769 to Barkey, Jr., et al., discloses protecting skin areas from irritation due to contact with artificial devices such as prosthetics, bandages and casts by applying a cyanoacrylate polymer to the desired skin areas that otherwise would be prone to ulceration or irritation by the devices.

A concern in the area of use of such adhesive compositions, is how to remove the composition when desired. In the area of medical grade adhesives, it may be especially desirable to remove the adhesive prior to the end of its usual lifetime. For example, it may be necessary to remove the adhesive composition from areas where the adhesive is not desired such as by misapplication or over application, from areas where an infection occurs underneath the adhesive, or from areas where the underlying tissue or wound needs to be re-accessed.

Various remover compositions have been developed for removing contaminants and/or adhesives. Among other materials, many industrial remover formulations contain isopropyl myristate or related substances. For instance, U.S. Pat. No. 5,849,106 discloses an industrial cleaning method for removing a contaminant comprising solder flux and/or adhesive tape residue from a substrate such as, for example, a printed circuit board. The process comprises applying a terpene-based cleaning composition to the substrate to solubilize contaminant present on the substrate, and then removing the solubilized contaminant using heated water. In one embodiment, the patent defines the cleaning composition as one that consists essentially of a substantially water-insoluble alkyl ester, wherein the alkyl ester can be isopropyl myristate.

In addition, U.S. Pat. No. 5,961,731 discloses a method for removing deleterious deposits from a surface. More specifically, the method is directed to removing a scuff mark from a shoe surface by applying a fingernail polish remover composition to a shoe surface with a cotton ball, rubbing the composition into the surface, and removing the scuff mark and the composition from the surface. The remover composition is described as consisting essentially of various materials including isopropyl myristate.

Another reference, Japanese Patent No. 10036227A2, discloses a nail enamel remover. The remover more easily removes water-based nail enamel. The remover comprises various materials including an ester branched alcohol, which can be selected from one or more of isopropyl myristate, 2-ethylhexyl palmitate and isobutyl octanoate.

In addition, various remover formulations have been developed for removing adhesives including, but not limited to, cyanoacrylate adhesives. For instance, U.S. Pat. No. 4,422,883 discloses a method for removing cyanoacrylate adhesives from surfaces. More specifically, the method comprises removing cyanoacrylate adhesives using a remover composition comprising acetonitrile as the active solvent.

U.S. Pat. No. 4,381,248 also discloses a composition for removing cyanoacrylate adhesives from surfaces. In this case, the remover composition contains acetonitrile, water, sodium carbonate, a surfactant and a filler such as cellulose, starch, bentonite, silica or aluminum octanoate.

Although known remover compositions and methods including, but not limited to, the above-described compositions and methods, are suitable for removing contaminants and/or adhesives, including cyanoacrylate adhesives, in industrial applications, there remains a need for a suitable remover composition and method for removing medical grade adhesive compositions. More specifically, because known remover compositions, including those described above, contain various solvents such as petroleum distillates or other additives, these remover compositions are not suitable for contact with open wounds, sores or tissue areas, including areas of compromised skin. As a result, these compositions are not suitable for removing medical grade adhesive compositions that are typically applied to open wounds, sores or otherwise compromised skin areas.

### SUMMARY OF THE INVENTION

The present invention provides a bio-compatible remover composition that is suitable for removing medical grade and other adhesive compositions including, but not limited to, 1,1-disubstituted ethylene monomers, such as cyanoacrylate adhesive compositions, and particularly those that are applied to wounds, sores or tissue areas including compromised skin areas. The remover composition of the present invention includes mainly a solvent, plasticizer or mixture thereof, preferably a bio-compatible solvent and/or plasticizer such as an alkylester or siloxane plasticizer, and an antimicrobial preservative to render the composition resistant to microorganisms

The present invention is also directed to methods of making such a remover composition.

The present invention is also directed to methods of using such a remover composition for removing medical grade and other adhesives from surfaces such as, for example, areas of tissue, including compromised areas of skin, by applying the remover composition to an adhesive composition present on a surface, and then removing the adhesive and the applied remover composition after the remover and adhesive composition have substantially reacted and/or interacted.

The present invention is also directed to a kit comprising at least an amount of the remover composition and an applicator for applying the composition to a surface where an adhesive composition is present. The kit can also include an amount of the adhesive composition itself.

### DETAILED DESCRIPTION OF PREFERRED EMBODIMENTS

This invention is directed to an adhesive remover composition, preferably a bio-compatible remover composition, methods of making such a composition, methods of using such a composition for removing medical grade and other adhesives by applying the composition to such an adhesive, and kits comprising the remover composition and an applicator to apply the remover composition and/or an adhesive material.

According to embodiments of the present invention, the remover composition contains any suitable solvent or plasticizer that effectively dissolves or plasticizes the adhesive material to be removed, and/or reduces the tackiness of the adhesive material such that it can be readily removed from the application site. As used herein, the term “remover” or “remover compound” is thus used to refer to compounds such as solvents or the like, which tend to dissolve the adhesive composition and thus break down its physical structure, as well as to plasticizers or the like, which tend to plasticize the adhesive composition and/or reduces the tackiness of the adhesive material, such that the adhesive composition can be readily removed from the application site. The remover composition can thus include either one or the other type of compound, or a combination thereof, to provide desired removal characteristics.

Although not required in all embodiments, the remover composition of the present invention is preferably bio-compatible. The term “biocompatible”, as used herein, means that the composition is suitable for application to tissue or skin, whether compromised or not, without causing any adverse affects. In addition, the remover composition preferably, although not necessary in all embodiments, includes a suitable preservative agent.

The compositions of the present invention include a suitable remover compound. Suitable remover compounds include, but are not limited to, various petroleum-based and natural-based oils, various surfactants, antibiotic ointments, plasticizers, cosmetic lotions, creams, and balms containing the above materials, mixtures thereof, and the like. Mixtures of one or more remover compounds can be used, if desired.

Suitable petroleum-based and natural-based oils include, but are not limited to, linseed oil, cotton seed oil, poppy seed oil, peanut oil, mineral oil, sesame oil, glycerin, petrolatum jelly, petrolatum, WD-40™, sodium lauryl sulfate, and the like. Suitable surfactants include but are not limited to Tween 20™, Tween 80™, Tween 100 ™ and other commercial surfactants that are well known in the art.

According to embodiments of the present invention, the remover composition preferably contains at least one plasticizer as, or as one component of, the remover compound. Suitable plasticizers include, for example, a suitable fatty acid ester, or more particularly an alkylester plasticizer, cocoa butter or synthetic equivalents such as similar triacylglycerol compositions, or carnauba wax. Examples of such materials include, but are not limited to, fatty acid esters commonly used in the cosmetic and pharmaceutical arts as emollients, plasticizers, lubricants, and the like. Particularly preferred suitable fatty acid esters include, but are not limited to, fatty acid esters formed from fatty acids and alcohols, where the fatty acid ester has a combined carbon number of at least 5 or 6, such as from about 7 or 8 to about 60, preferably from about 12 or 13 to about 40 and more preferably from about 15 to about 30. Thus, for example, suitable fatty acid esters include those formed from fatty acids having a carbon chain length of from about 2 to about 30, preferably from about 4 to about 22, including but not limited to acetic acid, butyric acid, lauric acid, palmitic acid, myristic acid, stearic acid, and the like. Suitable alcohols useful in forming the fatty acid esters include, but are not limited to, alcohols having a carbon chain length of from about 2 to about 30, preferably from about 4 to about 22, including but not limited to butanol, octanol, lauryl alcohol (dodecanol or dodecyl alcohol), cetyl alcohol, stearyl alcohol (octadecyl alcohol), and the like. Accordingly, suitable fatty acid esters include, but are not limited to, compounds represented by the following general formula (III):

CₚH₂ₚ₊₁COOC_{q}H_{2q+1} (III)

where p and q independently represent integers from about 2 to about 30, preferably from about 4 to about 22, and p+q represents an integer of 4 or more, such as from about 4 or about 5 to about 60, preferably from about 6 or about 7 to about 40, more preferably from about 8 or about 9 to about 30. In embodiments, p+q is may also range from about 10 or about 11 to about 30. In other embodiments of the present invention, such as where a longer chain fatty acid ester is desired, p+q represents an integer of from about 12 to about 60, preferably from about 13 to about 40, more preferably from about 15 to about 30. Particularly preferred fatty acid esters are octyl acetate (formula (III) where p=7, q=1), dodecyl acetate (formula (III) where p=11, q=1), such as EXXATE™ 1200 available from Exxon Corporation, octyl isononanoate, such as the KESSCO® octyl isononanoate products available from Stepan Company, isopropyl palmitate, and isopropyl myristate, such as the KESSCO® IPM NF products available from Stepan Company.

Other suitable plasticizers also include various polydimethylsiloxanes, such as octamethylcyclotetrasiloxane. Such additives are disclosed in, for example, U.S. Pat. No. 6,183,593 B1 issued Feb. 6, 2001, the entire disclosure of which is incorporated herein by reference.

In addition, other embodiments of suitable remover compositions of the present invention can include solids dissolved in suitable carrier solvents. Suitable solids include, but are not limited to, cetyl palmitate (cetin), such as the KESSCO® 653 product, available from Stepan Company, Northfield, Ill.

In preferred embodiments, the remover composition of the present invention includes octyl acetate or dodecyl acetate where shorter chain fatty acid esters are desired, or isopropyl myristate or isopropyl palmitate where longer chain fatty acid esters are desired, as the plasticizer.

When two or more remover compounds are used in combination in embodiments of the present invention, they can be used in any suitable proportions to provide the desired adhesive removal, use, or characteristics. Thus, for example, where two remover compounds are used in combination, the relative proportions of the compounds can vary in any suitable range of, by way of example only, from about 1:200 or 1:100 to about 100:1 or 200:1 by weight. For example, two remover compounds can be used in combination in a proportion of about 1:50 or 1:20 or 1:10 to about 10:1 or 20:1 or 50:1. In embodiments, a proportion of about 1:1 of two remover compounds may provide desirable remover compositions having properties of both of the remover compounds. Of course, the present invention is in no way limited to these ranges, as it will be understood that desired proportions can be selected based on the particular remover compounds being used and/or the characteristics sought to be achieved.

Furthermore, when two or more remover compounds are used in combination in embodiments of the present invention, it may be desirable to use one or more remover compounds that act as an adhesive remover (i.e., which dissolved the adhesive composition) in combination with one or more remover compounds that act as plasticizers (i.e., which reduce the tackiness of the adhesive). Such a combination can provide adhesive removal by both attacking the structure as well as the strength of the adhesive.

The remover composition of the present invention can also include one or more optional additives. Suitable additives can include, for example, but are not limited to, preservatives, solvents, skin softeners, skin antioxidants, fragrances, colorants, and the like. Moreover, although these additional materials may be present in the remover composition, they are generally only present in very small amounts such as, for example, from about 1% or less to about 15% by weight or less, preferably from about 1% or less to about 5%, or from about 5% or more to about 15%.

For example, incorporation of a fragrance may be particularly desirable in embodiments to mask the otherwise unpleasant odor of typical fatty acid esters. For example, some fatty acid esters may exhibit an odor, which can be masked by incorporating a suitable fragrance, such as a green apple fragrance, into the composition.

An antimicrobial preservative can be included in the composition to protect the composition against microorganisms and to prolong the storage life of the composition. Suitable preservatives include any of the known and commercially available preservatives, including the preservatives that can suitably be added to the adhesive composition itself. Examples of suitable preservatives include, but are not limited to, allantoin; various parabens and their mixtures such as methyl paraben; the various preservatives disclosed in U.S. patent application Ser. No. 09/430,180, the entire disclosure of which is incorporated herein by reference; acidic and phenolic preservatives such as benzoic acid and salts thereof, sorbic acid and salts thereof, propionic acid and salts thereof, boric acid and salts thereof, dehydroacetic acid and salts thereof, sulphurous and vanillic acid, phenol, cresol, chlorocresol, o-phenylphenol, chlorothymol, parabens, alkyl esters of parahydroxybenzoic acid, methyl, ethyl, propyl, benzyl and butyl-p-hydroxybenzoates; mercurial preservatives such as thiomersal, phenylmercuric acetate and nitrate, nitromersol, and sodium ethylmercurithiosalicylate; quaternary ammonium preservative compounds such as benzalkonium chloride, cetylpyridinium chloride, benzethonium chloride, and cetyltrimethyl ammonium bromide; and other compounds exhibiting preservative effects such as alcohols, chlorobutanol, phenoxy-2-ethanol, benzyl alcohol, β-phenylethyl alcohol, chlorohexidine, chloroform, 6-acettoxy-2,4-dimethyl-m-dioxane 2,4,4′ trichloro-2′-hydroxy-diphenylether, imidizolidinyl urea compound, bromo-2-nitropropanediol-1,3,5 bromo-5-nitrol-1,3 dioxane, 2-methyl-4-isothiazolin-3-one, 5 chloro derivative, 1-(3-chloroallyl)-3,5,7-triazo 1-azoniaadamantane, chloride (Dowicil 200)®; and the like. Mixtures of two or more antimicrobial preservatives can also be used, such as in the form of a preservative system, in embodiments, as desired. Other suitable preservatives are listed in Block, Seymour S., Disinfection, sterilization and Preservation, 4^{th} Ed., Lea and Feblger, Philadelphia, Pa., Tables 50-2 and 52-2, pp. 876, 904-905 (1991), the entire disclosure of which is incorporated herein by reference. In suitable embodiments of the present invention, one or more preservatives including, but not limited to the above-listed preservatives, can be combined.

In embodiments, suitable preservatives also include those available from International Specialty Products, such as GERMALL® 115 (imidazolidinyl urea), GERMALL® II (diazolidinyl urea), GERMALL® PLUS (99% GERMALL® II and 1% iodopropynylbutyl carbamate), GERMABEN® II (30% GERMALL® II, 11% methyl paraben, 3% propyl paraben, 56% propylene glycol), GERMABEN® II-E (20% GERMALL® II, 10% methyl paraben, 10% propyl paraben, 60% propylene glycol), SUTTOCIDE® A (50% aqueous solution of sodium hydroxymethylglyconate), LIQUIPAR® OIL (blend of isopropyl, isobutyl, and n-butyl esters of para-hydroxybenzoic acid), and LIQUIPAR® PE (blend of phenoxyethanol, isopropyl paraben, isobutyl paraben, and butyl paraben).

A suitable solvent can be included in the composition, if desired, to assist in application, action and/or removal of the remover composition and/or adhesive composition. Suitable solvents include, but are not limited to, acetone, ethyl acrylate, butyl acrylate, methyl acrylate, turpentine, alcohols including, but not limited to, ethanol, methanol, butanol, isopropyl alcohol, dimethylsulfoxide (DMSO) and methyl sulfone, dimethyl sulfone, mixtures thereof, and the like.

As mentioned above, the remover composition can also include one or more skin softeners, skin antioxidants, fragrances, colorants, and the like. Various suitable materials for each of these groups of additives are well known and/or would be readily available to one of ordinary skill in the art based on the present disclosure. Although there are no specific limitations on the types of additives used in the present invention, in preferred embodiments, the above additives are bio-compatible.

As also mentioned above, the remover composition of the present invention is preferably bio-compatible. As such, the composition preferably does not include any components that would be harmful to living tissue. For example, the remover composition is preferably free of, or at least substantially free of to the point that any adverse effects are minimal or unobservable, such non-bio-compatible substances as petroleum distillates.

Although not limited to any particular amounts, the remover composition of the present invention preferably includes the remover compound in a major amount of at least 50% by weight. In embodiments, the remover compound is preferably contained in an amount of at least 60% or at least 70% by weight, and more preferably in an amount of at least 80 or at least 90% by weight. Remover compositions having the remover compound present in an amount of at least 98% or at least 99% by weight are particularly preferred.

Preferably, the remover composition of the present invention contains as few components as possible. Thus, for example, in embodiments of the present invention, the remover composition consists essentially of, or even consists of, only the remover compound(s) and a preservative, with an optional fragrance. For example, the composition can consist essentially of or even consist of about 98% or more or about 99% or more by weight remover agent and from less than or about 1% to about less than or about 2% by weight preservative. A fragrance can also be included in suitable amounts, as desired. Of course, other ratios of components can also be used, in embodiments, as desired.

In addition, because the remover composition of the present invention in embodiments includes an antimicrobial preservative to protect the composition against microorganisms, the remover composition does not need to be sterilized during manufacture or packaging or before being applied to a compromised skin area. As a result, the use of a preservative in the remover composition of the present invention eliminates the added cost and time that would otherwise be required to properly sterilize the remover to render the composition suitable for contact with wounds, sores or compromised areas of skin. Generally, the preservative in the remover composition of the present invention is present in an amount sufficient to provide a Sterility Assurance Level (SAL) of at least 10⁻³. In embodiments, the Sterility Assurance Level may be at least 10⁻⁴, or may be at least 10⁻⁵, or may be at least 10⁻⁶.

In addition to, or instead of, exhibiting the above Sterility Assurance Level, in embodiments, the preservative in the remover composition of the present invention may be present in an amount sufficient to provide antimicrobial properties. Generally, the preservative in the remover composition of the present invention is present in an amount that is sufficient to provide antimicrobial properties that are at a level sufficient to render the composition suitable for contact with wounds, sores or compromised areas of skin. Although the preservative in the composition of the present invention may be present in varying amounts, the preservative is preferably present in an amount suitable to provide an effective microbial kill rate to pass appropriate or required preservative efficacy testing requirements as may be required for the particular use of the product. The antimicrobial properties of the composition of the present invention can be tested by a number of different methods including, for example, using the antimicrobial effectiveness testing procedures set forth in Section 51 of The United States Pharmacopeia, The National Formulary, pages 1809-1811 (January 2000) (USP), prepared by the United States Pharmacopeial Convention, Inc. at 12601 Twinbrook Parkway, Rockville, Md. 20852, the disclosure of which is incorporated herein by reference in its entirety. In addition, the number of microorganisms remaining in the composition can be estimated using the techniques set forth in Section 1227 of the USP, the disclosure of which is incorporated herein by reference in its entirety. That is, by using the methods described in Section 1227 of the USP, the tests described in Section 51 of the USP for determining antimicrobial effectiveness can be validated.

In embodiments, the remover composition of the present invention is capable of assisting in the removal of an adhesive composition, adhesive tape residue, other industrial adhesives, and mixtures thereof, from an application site. Adhesive materials that can be removed using the remover composition of the present invention include, but are not limited to, those adhesives that are formed from a polymerizable monomer (including prepolymeric) adhesive composition that comprises one or more polymerizable monomers. For example, the adhesive can be one that is formed from monomers that are readily polymerizable, e.g. anionically polymerizable or free radical polymerizable, or polymerizable by zwitterions or ion pairs to form polymers. Such monomers include those that form polymers, that may, but do not need to, biodegrade. Such monomers are disclosed in, for example, U.S. Pat. Nos. 5,328,687 and 5,928,611 to Leung et al., U.S. patent application Ser. No. 09/430,177, filed on Oct. 29, 1999, and U.S. patent application Ser. No. 09/471,392 filed Dec. 23, 1999, which are hereby incorporated in their entirety by reference herein. Preferred monomers include 1,1-disubstituted ethylene monomers, such as α-cyanoacrylates including, but not limited to, alkyl α-cyanoacrylates having an alkyl chain length of from about 1 to about 20 carbon atoms or more, preferably from about 2 to about 12 or more preferably from about 3 to about 8 carbon atoms. The α-cyanoacrylates can be prepared according to several methods known in the art. U.S. Pat. Nos. 2,721,858, 3,254,111, 3,995,641, and 4,364,876, each of which is hereby incorporated in its entirety by reference herein, disclose methods for preparing α-cyanoacrylates.

The present invention is also directed to methods of making the above-described remover composition. Methods of making the composition, according to the invention, include selecting a suitable remover compound, such as those described above, and combining the remover compound with one or more optional additives such as a suitable antimicrobial preservative, fragrance, and the like. The remover composition can be prepared by combining the plasticizer and preservative in various weight percent amounts. Generally, the method of making the remover composition of the present invention comprises mixing the remover compound and other ingredients.

The present invention is also directed to methods of using the remover composition for removing medical grade and other adhesives. Generally, the methods of the present invention for removing adhesives comprise applying the remover composition to an adhesive composition present on a surface such as, for example, tissue areas, including areas of compromised skin, using an applicator, and then removing the adhesive and the applied remover composition from the surface. Once the remover is applied, the adhesive and the remover can either be removed immediately after the remover composition is applied, or the remover composition can be maintained in contact with the adhesive for a set period of time to allow the remover composition to detackify the adhesive. Preferably, when the remover composition is not immediately removed, it is left in contact with the adhesive for up to about 1 minute, and preferably up to about 5 minutes, or more. For example, in one embodiment, the remover composition can be applied to a set adhesive composition and maintained in contact with the set adhesive composition for about 5 to 10 minutes, which time allows the remover composition to act on the adhesive composition and permit easy removal.

In embodiments of the present invention, various applicators and application methods can be used to apply the remover composition. Suitable applicators include, but are not limited to, spray applicators, sponge applicators, composition-soaked towelettes, brush applicators, swab applicators, and the like.

In an exemplary embodiment of the present invention a spray applicator may be preferred. A spray applicator, for instance, may be preferred when a large area of adhesive composition is to be removed from a surface. A spray applicator can conveniently apply a large amount of remover composition over a large area in a relatively uniform and controlled manner. Likewise, a spray applicator may be preferred in certain environments, such as in a hospital or clinic, where the remover may be repeatedly and frequently used.

In another exemplary embodiment, a towelette-type applicator may be preferred. The towelette may be prepared as a conventional soaked towelette, where the towelette is soaked with the remover composition, and sealed in a suitable container to prevent evaporation and loss of the remover composition. A suitable container can include, for example, a foil pouch and the like. A towelette applicator may be preferred in instances where a relatively small amount of remover composition is to be accurately applied to a specific skin area. For example, in instances where it is desirable to apply remover without inadvertently applying remover to unintended skin areas, a towelette applicator may provide greater control and, therefore, may be preferred instead of a spray applicator. Furthermore, a towelette applicator may be preferred where it is desirable to package the remover composition in convenient, single-use amounts. In addition, a towelette applicator can be used not only to apply the remover composition, but also to remove the applied remover composition and the adhesive after the remover has been applied to the adhesive.

Preferably, the remover composition is applied in a sufficient amount to entirely cover the desired area, which generally would correspond to an area where an adhesive composition, or a portion thereof, is present. In embodiments, the remover composition covers an additional area around the desired area, for example, to ensure that all of the adhesive composition is sufficiently removed.

The present invention is also directed to a kit that can include the above-described remover composition. In embodiments, the kit includes the remover composition, preferably with one or more applicator devices and/or an amount of adhesive composition, adhesive tape, other industrial adhesive, or mixtures thereof, that the remover composition is capable of removing. Such kits can be packaged in any suitable packing including, for example but not limited to, a box, a carton, a molded container, a unitary apparatus including all of the components, such as a two-sided applicator having adhesive on one side and removing on the other, shrink-wrap packaging, and the like.

For example, exemplary kits according to the present invention can include one or more adhesive applicators containing adhesive material, and one or more amounts of the remover composition. For example, such a kit can include one or more single-use ampoules of adhesive, and one or more preferably separately packaged towelettes moistened with the remover composition or one or more spray applicators containing the remover composition. Another exemplary kit can include, for example, a supply of adhesive material in a container, one or more applicators for applying the adhesive material, and a separate remover composition. The remover composition can be provided as, for example, one or more moistened towelettes, a spray container, or as a supply of remover composition in a container and one or more applicators for applying the remover composition.

The remover composition of the present invention can be used to remove a wide range of adhesive materials, but they are more preferably used to remove adhesive compositions that are derived from 1,1-disubstituted ethylene monomers, as described above. Thus, where the kit includes an amount of adhesive, it is preferred that the adhesive composition be such an adhesive as described above.

### EXAMPLES

### Example 1

A remover composition is prepared by combining isopropyl myristate and methylparaben in a weight percent ratio of 99% isopropyl myristate to 1% methylparaben. The mixture is sealed in a glass vial and stirred.

The characteristics of the composition are observed at about one minute after preparation and at twenty-four or more hours after preparation. The results of the observations show that the remover composition is substantially free from microorganism contamination. As a result no additional sterilization is required to prepare the composition for application to compromised skin areas where open wounds or sores may be present.

To test the removal effectiveness of the remover composition, two samples of a medical grade cyanoacrylate adhesive composition are applied to healthy skin areas, and the adhesive is allowed to polymerize. Attempts at removal of the first area of adhesive by rubbing and abrasion are negative. The remover composition is applied to the second area of adhesive using a moist towelette applicator. The amount of remover composition applied is sufficient to cover the entire area of the adhesive. After applying the remover composition to the adhesive, about 5 minutes are allowed to pass so that the remover has sufficient time to substantially interact with the adhesive. The remover and the adhesive composition are then removed by mild wiping using the moist towelette.

### Example 2

A remover composition is prepared by combining 99 wt % of a 1:1 mixture of dodecyl acetate and octamethylcyclotetrasiloxane with 1% methylparaben. A suitable amount of a green apple fragrance is added to the mixture. An amount of the mixture is sealed in a glass vial and stirred. Another amount of the composition is applied to a dry pad to form a towelette moistened with the mixture.

The characteristics of the composition in the sealed tube are observed at about one minute after preparation and at twenty-four or more hours after preparation. The results of the observations show that the remover composition is substantially free from microorganism contamination. As a result no additional sterilization is required to prepare the composition for application to compromised skin areas where open wounds or sores may be present.

To test the removal effectiveness of the remover composition, two samples of a medical grade cyanoacrylate adhesive composition are applied to healthy skin areas, and the adhesive is allowed to polymerize. Attempts at removal of the first area of adhesive by rubbing and abrasion are negative. The remover composition is applied to the second area of adhesive by placing the moistened towelette in contact with the adhesive. After applying the remover composition to the adhesive, about 10 minutes are allowed to pass so that the remover has sufficient time to substantially interact with the adhesive. The remover and the adhesive composition are then removed by mild wiping using the moistened towelette.

While the invention has been described with reference to preferred embodiments, the invention is not limited to the specific examples given, and other embodiments and modifications can be made by those skilled in the art without departing from the spirit and scope of the invention.

What is claimed is:

1. A bio-compatible adhesive remover composition, comprising:
one or more fatty acid esters having a carbon number of at least 5; and
one or more antimicrobial preservatives.

2. The composition of claim 1, wherein the fatty acid ester has the general formula:
CₚH₂ₚ₊₁COOC_{q}H_{2q+1}
wherein p and q independently represent integers of from about 2 to about 30, and p+q represents an integer of from about 4 to about 60.

3. The composition of claim 1, wherein the fatty acid ester has the general formula:
CₚH₂ₚ₊₁COOC_{q}H_{2q+1}
wherein p and q independently represent integers of from about 2 to about 30, and p+q represents an integer of from about 7 to about 60.

4. The composition of claim 1, wherein the fatty acid ester is formed from (1) a fatty acid selected from the group consisting of acetic acid, butyric acid, lauric acid, palmitic acid, myristic acid, and stearic acid, and (2) an alcohol selected from the group consisting of butanol, isopropanol, octanol, lauryl alcohol, cetyl alcohol, and stearyl alcohol.

5. The composition of claim 1, wherein the fatty acid ester is an alkylester myristate.

6. The composition of claim 1, wherein the fatty acid ester is isopropyl myristate.

7. The composition of claim 1, wherein the fatty acid ester is an alkylester acetate.

8. The composition of claim 1, wherein the fatty acid ester is selected from the group consisting of octyl acetate and dodecyl acetate.

9. The composition of claim 1, further comprising one or more plasticizers that are different from said one or more fatty acid esters.

10. The composition of claim 9, wherein said one or more plasticizers comprises a polydimethylsiloxanes.

11. The composition of claim 9, wherein said one or more plasticizers comprises octamethylcyclotetrasiloxane.

12. The composition of claim 9, wherein a weight ratio of said one or more fatty acid esters to said one or more plasticizers is from about 10:1 to about 1:10.

13. The composition of claim 9, wherein a weight ratio of said one or more fatty acid esters to said one or more plasticizers is about 1:1.

14. The composition of claim 9, wherein said composition consists essentially of the fatty acid ester, the plasticizer, and the antimicrobial preservative.

15. The composition of claim 9, wherein said composition consists essentially of the fatty acid ester, the plasticizer, the antimicrobial preservative, and a fragrance.

16. The composition of claim 9, wherein said composition comprises an acetate, a polydimethylsiloxane, the antimicrobial preservative, and a fragrance.

17. The composition of claim 1, wherein the remover composition is substantially free of petroleum distillates.

18. The composition of claim 1, wherein the antimicrobial preservative is present in an amount to make said remover composition have a Sterility Assurance Level (SAL) of 10⁻³-10⁻⁶.

19. The composition of claim 1, wherein the antimicrobial preservative is selected from the group consisting of allantoin and a paraben.

20. The composition of claim 1, wherein the antimicrobial preservative is at least one alkyl paraben.

21. The composition of claim 1, further comprising at least one fragrance.

22. The composition of claim 1, where the at least one fragrance is a green apple fragrance.

23. The composition of claim 1, wherein the remover composition consists essentially of the fatty acid ester and the antimicrobial preservative.

24. The composition of claim 23, wherein the fatty acid ester is isopropyl myristate.

25. The composition of claim 1, wherein the remover composition consists essentially of from about 90 to about 100 percent by weight of the fatty acid ester and from about 0 to about 10 percent by weight of the antimicrobial preservative.

26. The composition of claim 25, wherein the fatty acid ester is isopropyl myristate.

27. The composition of claim 1, wherein the remover composition consists essentially of the fatty acid ester, the antimicrobial preservative, and a fragrance.

28. The composition of claim 27, wherein the fatty acid ester is an acetate.

29. The composition of claim 1, further comprising one or more additives selected from the group consisting of solvents, skin softeners, skin antioxidants, fragrances, and colorants.

30. The composition of claim 1, wherein the fatty acid ester is cocoa butter.

31. A method of removing an adhesive composition, comprising:
applying the bio-compatible adhesive remover composition of claim 1 to an adhesive composition previously applied to a surface; and
removing the remover composition and the adhesive from the surface.

32. The method of claim 31, wherein the remover composition is allowed to set in contact with the adhesive composition for a period of time before the remover composition and the adhesive are removed.

33. The method of claim 31, wherein the period of time is at least 1 minute.

34. The method of claim 31, wherein the remover composition further comprising one or more plasticizers that are different from said one or more fatty acid esters.

35. The method of claim 34, wherein said one or more plasticizers comprises a polydimethylsiloxanes.

36. The method of claim 34, wherein said one or more plasticizers comprises octamethylcyclotetrasiloxane.

37. The method of claim 34, wherein a weight ratio of said one or more fatty acid esters to said one or more plasticizers is from about 10:1 to about 1:10.

38. The method of claim 34, wherein said remover composition consists essentially of the fatty acid ester, the plasticizer, and the antimicrobial preservative.

39. The method of claim 34, wherein said remover composition consists essentially of the fatty acid ester, the plasticizer, the antimicrobial preservative, and a fragrance.

40. The method of claim 31, wherein the remover composition consists essentially of the fatty acid ester and the antimicrobial preservative.

41. The method of claim 40, wherein the fatty acid ester is isopropyl myristate.

42. The method of claim 31, wherein the remover composition consists essentially of from about 90 to about 100 percent by weight of the fatty acid ester and from about 0 to about 10 percent by weight of the antimicrobial preservative.

43. The method of claim 42, wherein the fatty acid ester is isopropyl myristate.

44. The method of claim 31, wherein the remover composition consists essentially of the fatty acid ester, the antimicrobial preservative, and a fragrance.

45. The method of claim 44, wherein the fatty acid ester is an acetate.

46. The method of claim 31, wherein the remover composition is applied to said adhesive using an applicator.

47. The method of claim 46, wherein the applicator is selected from the group consisting of a spray applicator, a sponge applicator, and a towelette applicator.

48. The method of claim 46, wherein the applicator comprises a towelette applicator that is pre-moistened with said remover composition.

49. The method of claim 31, wherein said surface is an area of tissue.

50. The method of claim 31, wherein said surface is an area of compromised skin.

51. The method of claim 31, wherein said adhesive is formed from a polymerizable 1,1-disubstituted ethylene monomer.

52. The method of claim 31, wherein said adhesive is formed from a polymerizable α-cyanoacrylate monomer.

53. The method of claim 31, wherein the remover composition is substantially free of petroleum distillates.

54. The method of claim 31, wherein said remover composition is applied in an amount sufficient to completely cover said adhesive composition.

55. The method of claim 31, wherein said preservative is present in an amount to make said remover composition have a Sterility Assurance Level (SAL) of 10⁻³-10⁻⁶.

56. A method of making the remover composition of claim 1, the method comprising combining the fatty acid ester and the antimicrobial preservative.

57. A kit comprising:
a supply of a polymerizable monomeric adhesive composition comprising a 1,1-disubstituted ethylene monomer that is polymerizable to form a polymeric adhesive; and
a bio-compatible adhesive remover composition capable of removing the polymeric adhesive.

58. The kit of claim 57, wherein the remover composition comprises:
a remover compound selected from the group consisting of petroleum-based oils, natural oils, glycerin, petrolatum jelly, petrolatum, WD-40, sodium lauryl sulfate, surfactants, and antibiotic ointments.

59. The kit of claim 58, wherein the remover compound is a natural oil selected from the group consisting of poppy seed oil, linseed oil, cotton seed oil, peanut oil, mineral oil, and sesame oil.

60. The kit of claim 57, wherein the remover composition comprises cocoa butter.

61. The kit of claim 57, wherein the remover composition further comprises an antimicrobial preservative.

62. The kit of claim 57, wherein the remover compound is a fatty acid ester having a carbon number of at least 5, and said remover composition further comprises an antimicrobial preservative.

63. The kit of claim 62, wherein the fatty acid ester has the general formula:
CₚH₂ₚ₊₁COOC_{q}H_{2q+1}
wherein p and q independently represent integers of from about 2 to about 30, and p+q represents an integer of from about 4 to about 60.

64. The kit of claim 62, wherein the fatty acid ester has the general formula:
CₚH₂ₚ₊₁COOC_{q}H_{2q+1}
wherein p and q independently represent integers of from about 2 to about 30, and p+q represents an integer of from about 7 to about 60.

65. The kit of claim 62, wherein the fatty acid ester is formed from (1) a fatty acid selected from the group consisting of acetic acid, butyric acid, lauric acid, palmitic acid, myristic acid and stearic acid, and (2) an alcohol selected from the group consisting of butanol, isopropanol, lauryl alcohol, cetyl alcohol, octanol, and stearyl alcohol.

66. The kit of claim 62, wherein the fatty acid ester is an alkylester myristate.

67. The kit of claim 62, wherein the fatty acid ester is isopropyl myristate.

68. The kit of claim 62, wherein the fatty acid ester is an alkylester acetate.

69. The kit of claim 62, wherein the fatty acid ester is selected from the group consisting of octyl acetate and dodecyl acetate.

70. The kit of claim 62, further comprising one or more plasticizers that are different from said one or more fatty acid esters.

71. The kit of claim 70, wherein said one or more plasticizers comprises a polydimethylsiloxanes.

72. The kit of claim 70, wherein said one or more plasticizers comprises octamethylcyclotetrasiloxane.

73. The kit of claim 70, wherein a weight ratio of said one or more fatty acid esters to said one or more plasticizers is from about 10:1 to about 1:10.

74. The kit of claim 70, wherein a weight ratio of said one or more fatty acid esters to said one or more plasticizers is about 1:1.

75. The kit of claim 70, wherein said composition consists essentially of the fatty acid ester, the plasticizer, and the antimicrobial preservative.

76. The kit of claim 70, wherein said composition consists essentially of the fatty acid ester, the plasticizer, the antimicrobial preservative, and a fragrance.

77. The kit of claim 70, wherein said composition comprises an acetate, a polydimethylsiloxane, the antimicrobial preservative, and a fragrance.

78. The kit of claim 62, wherein the remover composition is substantially free of petroleum distillates.

79. The kit of claim 62, wherein the antimicrobial preservative is present in an amount to make said remover composition have a Sterility Assurance Level (SAL) of 10⁻³-10⁻⁶.

80. The kit of claim 62, wherein the antimicrobial preservative is selected from the group consisting of allantoin and a paraben.

81. The kit of claim 62, wherein the antimicrobial preservative is at least one alkyl paraben.

82. The kit of claim 62, further comprising at least one fragrance.

83. The kit of claim 62, where the at least one fragrance is a green apple fragrance.

84. The kit of claim 62, wherein the remover composition consists essentially of the fatty acid ester and the antimicrobial preservative.

85. The kit of claim 84, wherein the fatty acid ester is isopropyl myristate.

86. The kit of claim 62, wherein the remover composition consists essentially of from about 90 to about 100 percent by weight of the fatty acid ester and from about 0 to about 10 percent by weight of the antimicrobial preservative.

87. The kit of claim 86, wherein the fatty acid ester is isopropyl myristate.

88. The kit of claim 62, wherein the remover composition consists essentially of the fatty acid ester, the antimicrobial preservative, and a fragrance.

89. The kit of claim 88, wherein the fatty acid ester is an acetate.

90. The kit of claim 62, wherein the remover composition further comprises one or more additives selected from the group consisting of solvents, skin softeners, skin antioxidants, fragrances, and colorants.

91. The kit of claim 57, further comprising an applicator for said remover composition.

92. The kit of claim 91, wherein said remover composition is stored separate from said application prior to use.

93. The kit of claim 91, wherein said remover composition is stored together and in contact with said applicator prior to use.

94. The kit of claim 91, wherein said applicator is selected from the group consisting of a spray applicator, a sponge applicator, and a towelette applicator.

95. The kit of claim 57, wherein said remover composition is absorbed or adsorbed in a towelette.

96. The kit of claim 95, wherein the towelette is stored in a sealed foil package.
